(19) United States
(12) Patent Application Publication
(10) Pub. No.: US 2016/0194352 A1
(43) Pub. Date: Jul. 7, 2016

(54) AMORPHOUS FORMS OF DACLATASVIR DIHYDROCHLORIDE

(71) Applicant: Dr.Reddy's Laboratories Ltd., Hyderabad (IN)

(72) Inventors: Venkata Narasayya SALADI, Hyderabad (IN); Vishweshwar PEDDY, Hyderabad (IN)

(21) Appl. No.: 14/988,372

(22) Filed: Jan. 5, 2016

(30) Foreign Application Priority Data
Jan. 5, 2015 (IN) 37/CHE/2015

(51) Int. Cl. C07K 5/062

(52) U.S. Cl. CPC C07K 5/06052; A61K 38/00

(57) ABSTRACT

The present application relates to the amorphous form of Daclatasvir dihydrochloride and the processes for the preparation thereof. The application further provides its solid dispersion having Daclatasvir dihydrochloride in amorphous form and process for its preparation.

This application claims the benefit of Indian Provisional Application No. 37/CHE/2015, filed Jan. 5, 2015, all of which are hereby incorporated by reference in their entireties.

### INTRODUCTION

The present application relates to the amorphous forms of daclatasvir dihydrochloride.

The present application related to solid dispersion comprising amorphous daclatasvir dihydrochloride.

Chemically Daclatasvir is named Dimethyl N,N′-(biphenyl-4,4′-diylbis{1H-imidazole-5,2-diyl-[(2 S)-pyrrolidine-2,1-diyl][(1 S)-1-(1-methylethyl)-2-oxoethane-2,1-diyl]})dicarbamate and daclatasvir dihydrochloride has the structural formula as shown in Formula I.

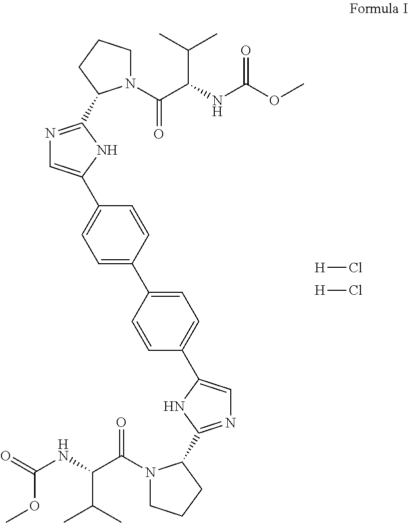

Daclatasvir (formerly BMS-790052, trade name Daklinza) is a drug for the treatment of hepatitis C (HCV). It was developed by Bristol Myers squib and was approved in Europe on 22 Aug. 2014. Daclatasvir inhibits the HCV nonstructural protein NS5A. Recent research suggests that it targets two steps of the viral replication process, enabling rapid decline of HCV RNA. Hepatitis C virus (HCV) is a major human pathogen, infecting an estimated 170 million persons worldwide roughly five times the number infected by human immunodeficiency virus type 1. A substantial fraction of these HCV infected individuals develop serious progressive liver disease, including cirrhosis and hepatocellular carcinoma. Presently, the most effective HCV therapy employs a combination of alpha-interferon and ribavirin, leading to sustained efficacy in 40 percent of patients. Recent clinical results demonstrate that pegylated alpha-interferon is superior to unmodified alpha-interferon as monotherapy. However, even with experimental therapeutic regimens involving combinations of pegylated alpha-interferon and ribavirin, a substantial fraction of patients do not have a sustained reduction in viral load. Thus, there is a clear and unmet need to develop effective therapeutics for treatment of HCV infection.

U.S. Pat. No. 7,728,027B2 discloses a process for the preparation of daclatasvir or a pharmaceutically acceptable salt thereof.

U.S. Pat. No. 8,629,171B2 discloses form N-2 of daclatasvir dihydrochloride which is characterized by (i) unit cell parameters, (ii) fractional atomic coordinates within the unit cell and (iii) characteristic peaks in the powder X-Ray diffraction pattern.

It has been disclosed earlier that the amorphous forms in a number of drugs exhibit different dissolution characteristics and in some cases different bioavailability patterns compared to crystalline forms [Konne T., Chem pharm Bull., 38, 2003(1990)]. For some therapeutic indications one bioavailability pattern may be favored over another. An amorphous form of Cefuroxime axetil is a good example for exhibiting higher bioavailability than the crystalline form.

There remains a need to provide solid state forms of daclatasvir dihydrochloride which have better physico-chemical properties in a cost effective and environment friendly manner.

### SUMMARY

In the first embodiment, the present application provides an amorphous form of daclatasvir dihydrochloride.

In the second embodiment, the present application provides an amorphous form of daclatasvir dihydrochloride characterized by powder X-ray diffraction (PXRD) pattern substantially as illustrated by FIGS. 1-5 respectively.

In the third embodiment the present application provides a process for preparing amorphous form of daclatasvir dihydrochloride, which comprises;

a) providing a solution of daclatasvir dihydrochloride in a solvent;
b) removing solvent from a solution of daclatasvir dihydrochloride obtained in step (a) and
c) recovering amorphous form of daclatasvir dihydrochloride.

In the fourth embodiment, the present application provides a process for preparing amorphous form of daclatasvir dihydrochloride, which comprises:

a) providing a solution of daclatasvir dihydrochloride in a solvent comprising methanol;
b) treating a solution obtained in step (a) with an anti-solvent and
c) recovering amorphous form of daclatasvir dihydrochloride.

In the fifth embodiment, the present application provides a solid dispersion comprising an amorphous form of daclatasvir dihydrochloride and one or more pharmaceutically acceptable carriers.

In the sixth embodiment, the present application provides a solid dispersion comprising an amorphous form of daclatasvir dihydrochloride and one or more pharmaceutically acceptable carriers characterized by powder X-ray diffraction (PXRD) substantially as illustrated by FIGS. 6-11 respectively.

In the seventh embodiment, the present application provides a process for preparing a solid dispersion comprising an amorphous form of daclatasvir dihydrochloride and one or more pharmaceutically acceptable carriers, which comprises;

a) providing a solution comprising daclatasvir dihydrochloride and one or more pharmaceutically acceptable carriers,
b) removing solvent from a solution obtained in step (a) and
c) recovering a solid dispersion comprising an amorphous form of daclatasvir dihydrochloride and one or more pharmaceutically acceptable carriers.

### BRIEF DESCRIPTION OF THE DRAWINGS

FIG. 1 is powder X-ray power diffraction (“PXRD”) pattern of amorphous form of daclatasvir dihydrochloride prepared according to Example 1.

FIG. 2 is powder X-ray power diffraction (“PXRD”) pattern of amorphous form of daclatasvir dihydrochloride prepared according to Example 2.

FIG. 3 is powder X-ray power diffraction (“PXRD”) pattern of amorphous form of daclatasvir dihydrochloride prepared according to Example 3.

FIG. 4 is powder X-ray power diffraction (“PXRD”) pattern of amorphous form of daclatasvir dihydrochloride prepared according to Example 4.

FIG. 5 is powder X-ray power diffraction (“PXRD”) pattern of amorphous form of daclatasvir dihydrochloride prepared according to Example 5.

FIG. 6 is powder X-ray power diffraction (“PXRD”) pattern of a solid dispersion comprising an amorphous form of daclatasvir dihydrochloride and copovidone prepared according to Example 6 (a).

FIG. 7 is powder X-ray power diffraction (“PXRD”) pattern of a solid dispersion comprising an amorphous form of daclatasvir dihydrochloride, copovidone and syloid prepared according to Example 6 (b).

FIG. 8 is powder X-ray power diffraction (“PXRD”) pattern of a solid dispersion comprising an amorphous form of daclatasvir dihydrochloride and PVP K-30 prepared according to Example 7 (a).

FIG. 9 is powder X-ray power diffraction (“PXRD”) pattern of a solid dispersion comprising an amorphous form of daclatasvir dihydrochloride, PVP K-30 and syloid prepared according to Example 7 (b).

FIG. 10 is powder X-ray power diffraction (“PXRD”) pattern of a solid dispersion comprising an amorphous form of daclatasvir dihydrochloride and HPC prepared according to Example 8 (a).

FIG. 11 is powder X-ray power diffraction (“PXRD”) pattern of a solid dispersion comprising an amorphous form of daclatasvir dihydrochloride, HPC and syloid prepared according to Example 8 (b).

### DETAILED DESCRIPTION

The present invention provides amorphous forms of daclatasvir dihydrochloride.

Daclatasvir dihydrochloride may be used as the input in the process for preparation of amorphous and amorphous solid dispersions of the present application can be prepared by any process known in the art.

In the first embodiment, the present application provides an amorphous form of daclatasvir dihydrochloride.

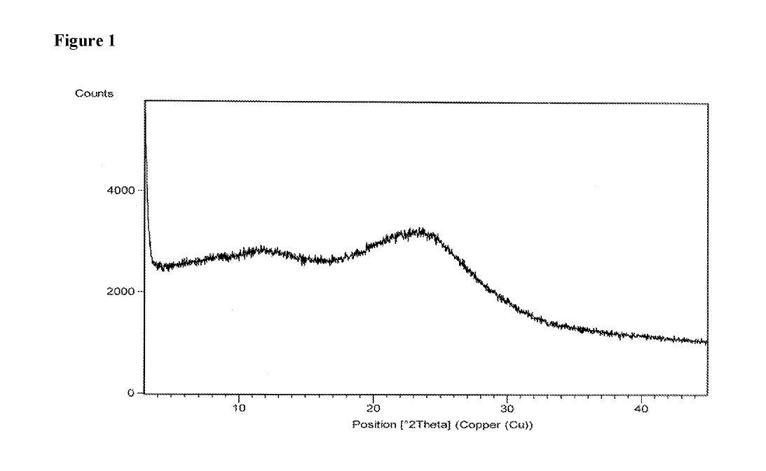

In the second embodiment, the present application provides an amorphous form of daclatasvir characterized by powder X-ray diffraction (PXRD) pattern substantially as illustrated by FIGS. 1-5 respectively.

In a first variant, the present application provides an amorphous form of daclatasvir characterized by powder X-ray diffraction (PXRD) pattern substantially as illustrated by FIG. 1.

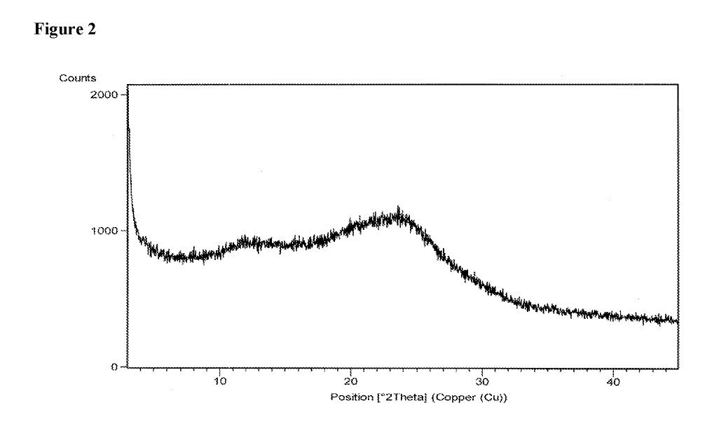

In a second variant, the present application provides an amorphous form of daclatasvir characterized by powder X-ray diffraction (PXRD) pattern substantially as illustrated by FIG. 2.

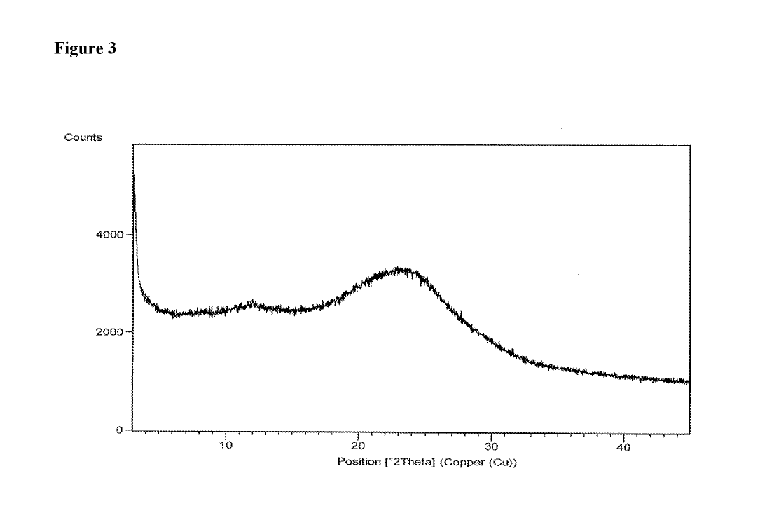

In a third variant, the present application provides an amorphous form of daclatasvir characterized by powder X-ray diffraction (PXRD) pattern substantially as illustrated by FIG. 3.

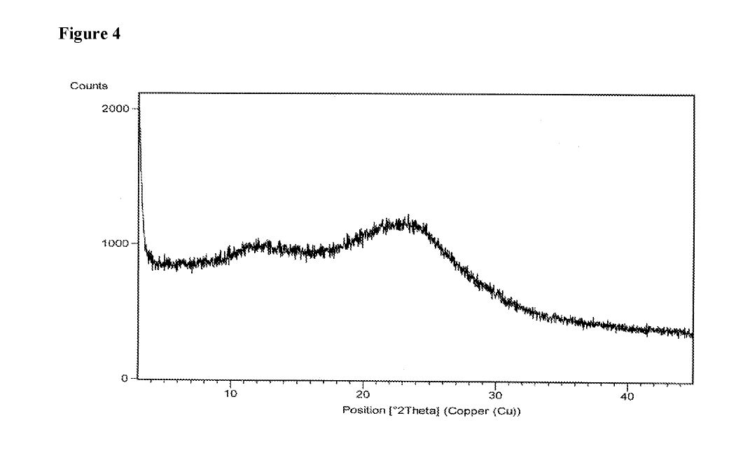

In a fourth variant, the present application provides an amorphous form of daclatasvir characterized by powder X-ray diffraction (PXRD) pattern substantially as illustrated by FIG. 4.

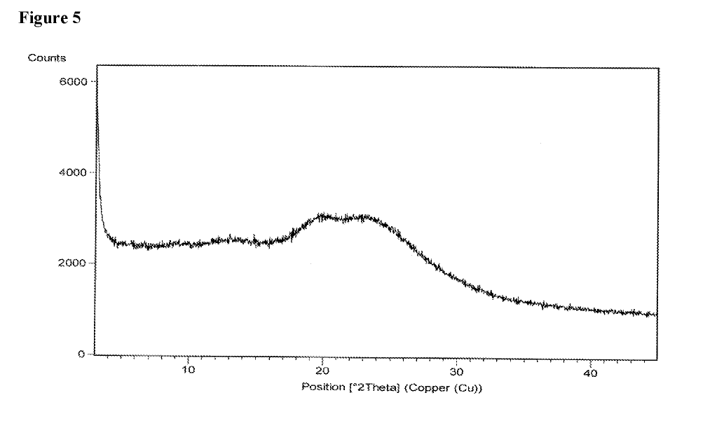

In a fifth variant, the present application provides an amorphous form of daclatasvir characterized by powder X-ray diffraction (PXRD) pattern substantially as illustrated by FIG. 5.

In the third embodiment, the present application provides a process for preparing amorphous form of daclatasvir dihydrochloride, which comprises;

a) providing a solution of daclatasvir dihydrochloride in a solvent;
b) removing solvent from a solution of daclatasvir dihydrochloride obtained in step a); and
c) recovering amorphous form of daclatasvir dihydrochloride.

Providing a solution in step a) includes:

i) direct use of a reaction mixture containing daclatasvir dihydrochloride that is obtained in the course of its synthesis; or ii) a solution that is obtained by treating daclatasvir with hydrogen chloride; or
ii) dissolving daclatasvir dihydrochloride in a solvent.

Any physical form of daclatasvir dihydrochloride may be utilized for providing the solution of daclatasvir dihydrochloride in step (a). Similarly any physical form of daclatasvir may be used as an input for providing a solution of daclatasvir dihydrochoride.

Suitable solvents which can be used for dissolving daclatasvir dihydrochloride include but are not limited to: water, dimethyl sulphoxide, dimethyl formamide, alcoholic solvents such as methanol, n-propanol, isoamyl alcohol and the like; halogenated hydrocarbons such as 1,2-dichloroethane, carbon tetrachloride and the like; esters such as n-propyl acetate, n-butyl acetate, t-butyl acetate and the like; ethers such as dimethyl ether, diisopropyl ether, and the like; hydrocarbons such as toluene and the like; nitriles such as propionitrile and the like; acids such as acetic acid, formic acid and any mixtures of two or more thereof.

After dissolution in step (a), the obtained solution may be optionally filtered to remove any insoluble particles. Suitable techniques to remove insoluble particles are filtration, centrifugation, decantation, and any other known techniques in the art. The solution can be filtered by passing through paper, glass fiber, or other membrane material, or a clarifying agent such as celite. Depending upon the equipment used and the concentration and temperature of the solution, the filtration apparatus may need to be preheated to avoid premature precipitation of solid.

Step (b) involves removing solvent from a solution of daclatasvir dihydrochloride obtained in step (a).

Suitable techniques which can be used for the removal of solvent include but not limited to evaporation, flash evaporation, simple evaporation, rotational drying, spray drying, agitated thin-film drying, agitated nutsche filter drying, pressure nutsche filter drying, freeze-drying or any other suitable technique known in the art.

Step (c) involves recovering an amorphous form of daclatasvir dihydrochloride. The said recovery can be by using the processes known in the art.

The resulting compound in step (c) may be optionally further dried. Drying can be carried out in a tray dryer, vacuum oven, air oven, cone vacuum dryer, rotary vacuum dryer, fluidized bed dryer, spin flash dryer, flash dryer, or the like.

The drying can be carried out at temperatures of less than about 60° C., less than about 50° C., less than about 40° C., less than about 30° C., less than about 20° C., or any other suitable temperatures; at atmospheric pressure or under a reduced pressure; as long as the daclatasvir dihydrochloride is not degraded in its quality. The drying can be carried out for any desired times until the required product quality is achieved. Suitable time for drying can vary from few minutes to several hours for example from about 30 minutes to about 24 or more hours.

In the fourth embodiment, the present application provides a process for preparing amorphous form of daclatasvir dihydrochloride, which comprises:

(a) providing a solution of daclatasvir dihydrochloride in a solvent comprising methanol;
(b) treating a solution obtained in step (a) with an anti-solvent and
(c) recovering amorphous form of daclatasvir dihydrochloride.

Suitable anti solvents which can be used for isolating the amorphous form of daclatasvir dihydrochloride include but are not limited to alcohol solvents, such as Isopropyl alcohol, ethanol, 1-butanol, 2-butanol and the like; Ketone solvents such as acetone, methyl ethyl ketone, Iso butyl methyl ketone, aromatic hydrocarbon solvents such as Xylene and the like; halogenated hydrocarbon solvents, such as dichloromethane, chloroform and the like; ester solvents such as methyl acetate, ethyl acetate, isopropyl acetate and the like; ether solvents, such as diethyl ether, 1,4-dioxane, tetrahydrofuran and the like; nitrile solvents such acetonitrile and the like; alkanes such as n-Heptane, n-Hexane and the like; or any mixtures thereof.

Surprisingly when a solution of daclatasvir dihydrochloride obtained in step (a) was added to an anti-solvent, the recovered amorphous form was found to be stable for 24 hrs when exposed to ambient atmosphere i.e., about 25° C. and about 55% RH (relative humidity).

In the fifth embodiment, the present application provides a solid dispersion comprising an amorphous form of daclatasvir dihydrochloride and one or more pharmaceutically acceptable carriers.

Solid dispersion as used herein refers to the dispersion of active ingredient in an inert excipient or matrix (carrier) or in multiple excipients, where the input active ingredients could exist in finely crystalline, solubilized or amorphous state (Sareen et al., 2012 and Kapoor et al., 2012). Solid dispersion consists of two or more than two components, generally a carrier polymer and drug optionally along with stabilizing agent (and/or surfactant or other additives). The most important role of the added polymer in solid dispersion is to reduce the molecular mobility of the drug to avoid the phase separation and re-crystallization of drug during storage. The increase in solubility of the drug in solid dispersion is mainly because drug remains in amorphous form which is associated with a higher energy state as compared to crystalline counterpart and due to that it required very less external energy to dissolve.

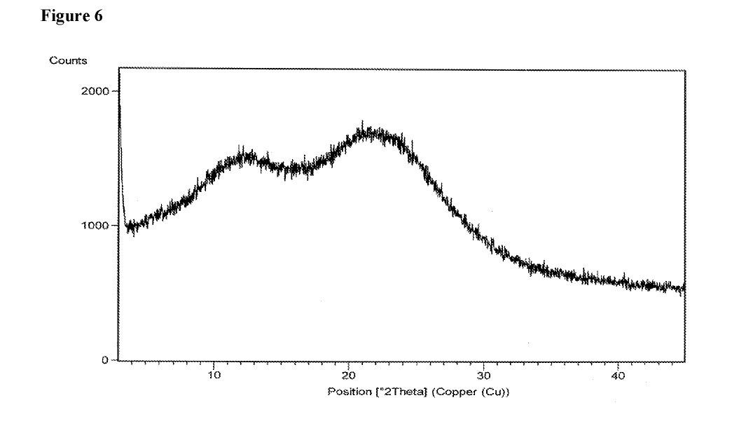

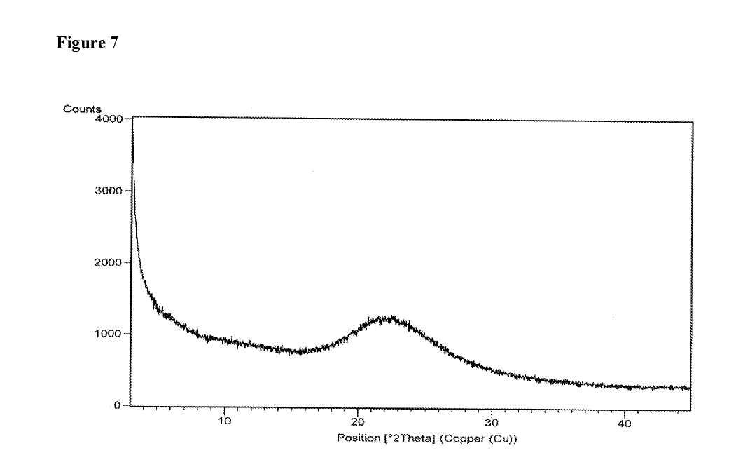

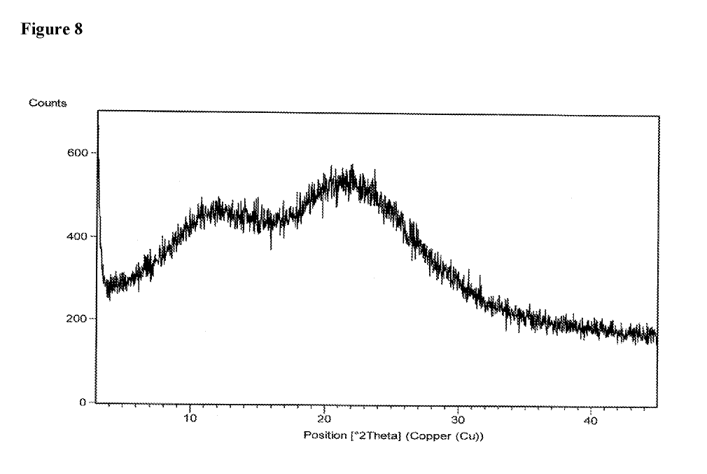

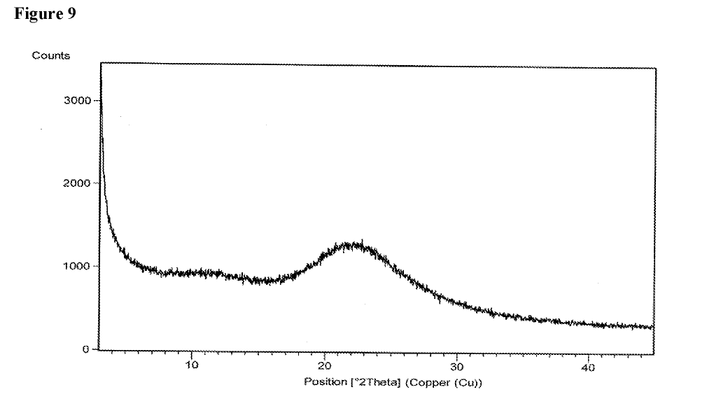

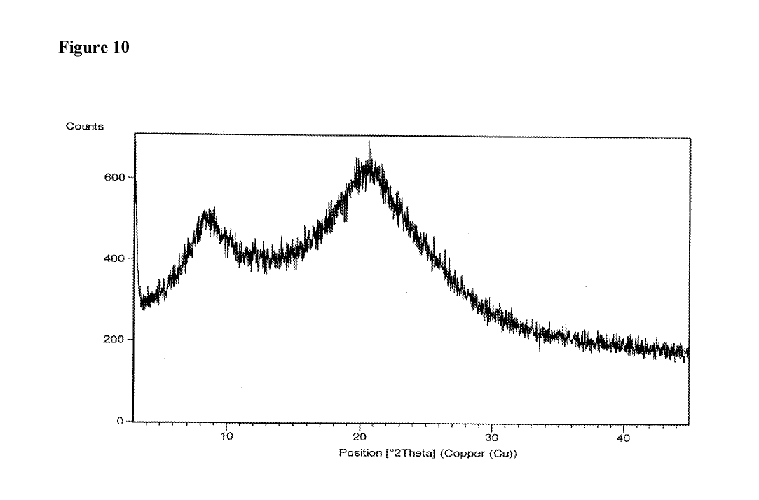

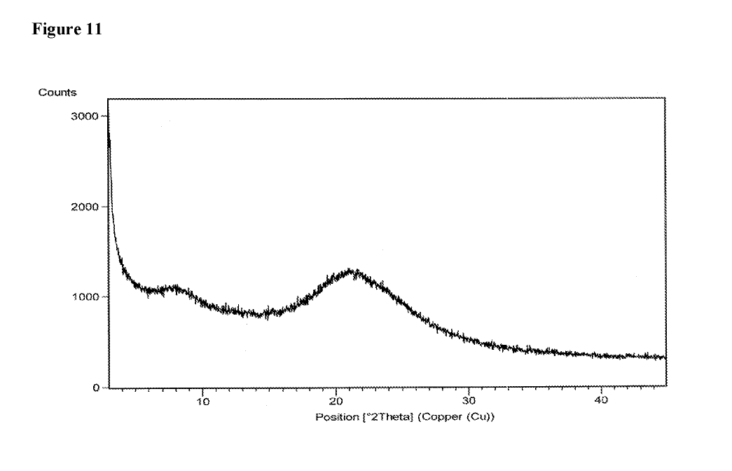

In the sixth embodiment, the present application provides a solid dispersion comprising an amorphous form of daclatasvir dihydrochloride and one or more pharmaceutically acceptable carriers characterized by powder X-ray diffraction (PXRD) substantially as illustrated by FIGS. 6-11 respectively.

In the seventh embodiment, the present application provides a process for preparing a solid dispersion comprising an amorphous form of daclatasvir dihydrochloride and one or more pharmaceutically acceptable carriers, which comprises;

a) providing a solution comprising daclatasvir dihydrochloride and one or more pharmaceutically acceptable carriers,
b) removing solvent from a solution obtained in step (a) and
c) recovering a solid dispersion comprising an amorphous form of daclatasvir dihydrochloride and one or more pharmaceutically acceptable carriers.

Providing a solution in step a) includes:

i) direct use of a reaction mixture containing daclatasvir dihydrochloride that is obtained in the course of its synthesis; or
ii) direct use of a reaction mixture containing daclatasvir dihydrochloride that is obtained by treating daclatasvir with hydrogen chloride; or
ii) dissolving daclatasvir dihydrochloride and pharmaceutically acceptable carrier in a solvent.

Any physical form of daclatasvir dihydrochloride may be utilized for providing the solution of daclatasvir dihydrochloride in step (a).

Suitable pharmaceutically acceptable carriers which can be used in step (a) include, but are not limited to: diluents such as starches, pregelatinized starches, lactose, powdered celluloses, microcrystalline celluloses, dicalcium phosphate, tricalcium phosphate, mannitol, sorbitol, sugar and the like; binders such as acacia, guar gum, tragacanth, gelatin, polyvinylpyrrolidones, copovidone, hydroxypropyl celluloses, hydroxypropyl methylcelluloses, pregelatinized starches and the like; disintegrants such as starches, sodium starch glycolate, pregelatinized starches, crospovidones, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, ethylcelluloses, various grades of methyl methacrylates, waxes and the like.

Other pharmaceutically acceptable excipients that are of use include but are not limited to film formers, plasticizers, colorants, flavoring agents, sweeteners, viscosity enhancers, syloids, preservatives, antioxidants, and the like.

Suitable solvents which can be used for dissolving the daclatasvir dihydrochloride include but are not limited to: alcoholic solvents such as methanol, n-propanol, isoamyl alcohol and the like; halogenated hydrocarbons such as 1,2-dichloroethane, carbon tetrachloride and the like; esters such as ethyl acetate, n-propyl acetate, n-butyl acetate, t-butyl acetate and the like; ethers such as dimethyl ether, diisopropyl ether and the like; hydrocarbons such as toluene and the like; nitriles such as propionitrile and the like; and any mixtures of two or more thereof.

After dissolution in step (a), optionally undissolved particles, if any, may be removed suitably by filtration, centrifugation, decantation, and any other known techniques. The solution can be filtered by passing through paper, glass fiber, or other membrane material, or a clarifying agent such as celite. Depending upon the equipment used and the concentration and temperature of the solution, the filtration apparatus may need to be preheated to avoid premature crystallization.

Step (b) involves removing solvent from a solution obtained in step (a);

Suitable techniques which can be used for the removal of solvent include but not limited to evaporation, flash evaporation, simple evaporation, rotational drying, spray drying, agitated thin-film drying, agitated nutsche filter drying, pressure nutsche filter drying, freeze-drying or any other technique known in the art.

Step (c) involves recovering a solid dispersion comprising an amorphous form of daclatasvir dihydrochloride and one or more pharmaceutically acceptable carriers. The said recovery can be by using the processes known in the art.

The resulting compound obtained in step (c) may be optionally further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 60° C., less than about 50° C., less than about 40° C., less than about 30° C., less than about 20° C., or any other suitable temperatures; at atmospheric pressure or under a reduced pressure; as long as the daclatasvir dihydrochloride is not degraded in its quality. The drying can be carried out for any desired times until the required product quality is achieved.

Suitable time for drying can vary from few minutes to several hours for example from about 30 minutes to about 24 or more hours.

In general, the solid states described herein may have particle size of less than about 200 μm, or less than about 150 μm, or less than about 100 μm, or less than about 90 μm, or less than about 80 μm, or less than about 60 μm, or less than about 50 μm, or less than about 40 μm, or less than about 30 μm, or less than about 20 μm, or less than about 10 μm, or less than about 5 μm, or any other suitable particle sizes.

Solid state forms of daclatasvir dihydrochloride of the present application are characterized by its PXRD pattern. All PXRD data reported herein were obtained using Cu Kα radiation, having the wavelength 1.541 Å, and were obtained using a PANalytical, Powder X-ray diffractometer.

Although the exemplified procedures herein illustrate the practice of the present invention in some of its embodiments, the procedures should not be construed as limiting the scope of the invention. Modifications from consideration of the specification and examples within the ambit of current scientific knowledge will be apparent to one skilled in the art.

### DEFINITIONS

The following definitions are used in connection with the present application unless the context indicates otherwise.

“Amorphous form” as used herein refers to a solid state wherein the amorphous content with in the said solid state is at least about 35% or at least about 40% or at least about 45% or at least about 50% or at least about 55% or at least about 60% or at least about 65% or at least about 70% or at least about 75% or at least about 80% or at least about 85% or at least about 90% or at least about 95% or at least about 96% or at least about 97% or at least about 98% or at least about 99% or about 100%.

An “alcohol” is an organic compound containing a carbon bound to a hydroxyl group. “C1-C6 alcohols” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentylalcohol, isoamyl alcohol, diethylene glycol monomethyl ether, diethylene glycol monoethylether, cyclohexanol, phenol, glycerol, or the like.

An “aliphatic hydrocarbon” is a liquid hydrocarbon compound, which may be linear, branched, or cyclic and may be saturated or have as many as two double bonds. A liquid hydrocarbon compound that contains a six-carbon group having three double bonds in a ring is called “aromatic.” Examples of “C5-C8 aliphatic or aromatic hydrocarbons” include, but are not limited to, isopentane, neopentane, n-hexane, isohexane, 3-methylpentane, 2,3-dimethylbutane, neohexane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, methylcyclohexane, cycloheptane, petroleum ethers, benzene toluene, ethylbenzene, m-xylene, o-xylene, p-xylene, trimethylbenzene, chlorobenzene, fluorobenzene, trifluorotoluene, anisole, or any mixtures thereof.

An “ester” is an organic compound containing a carboxyl group —(C═O)—O— bonded to two other carbon atoms. “C3-C6 esters” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, or the like.

An “ether” is an organic compound containing an oxygen atom —O— bonded to two other carbon atoms. “C2-C6 ethers” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, or the like.

A “halogenated hydrocarbon” is an organic compound containing a carbon bound to a halogen. Halogenated hydrocarbons include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

A “ketone” is an organic compound containing a carbonyl group —(C═O)— bonded to two other carbon atoms. “C3-C6 ketones” include, but are not limited to, acetone, ethyl methyl ketone, diethyl ketone, methyl isobutyl ketone, ketones, or the like.

A “polar aprotic solvent” has a dielectric constant greater than 15 and is at least one selected from the group consisting of amide-based organic solvents, such as N,N-dimethylformamide (DMF), N,N-dimethylacetamide (DMA), N-methylpyrrolidone (NMP), formamide, acetamide, propanamide, hexamethyl phosphoramide (HMPA), and hexamethyl phosphorus triamide (HMPT); nitro-based organic solvents, such as nitromethane, nitroethane, nitropropane, and nitrobenzene; pyridine-based organic solvents, such as pyridine and picoline; sulfone-based solvents, such as dimethylsulfone, diethylsulfone, diisopropylsulfone, 2-methylsulfolane, 3-methylsulfolane, 2,4-dimethylsulfolane, 3,4-dimethy sulfolane, 3-sulfolene, and sulfolane; and sulfoxide-based solvents such as dimethylsulfoxide (DMSO).

A “nitrile” is an organic compound containing a cyano —(C≡N) bonded to another carbon atom. “C2-C6 Nitriles” include, but are not limited to, acetonitrile, propionitrile, butanenitrile, or the like.

All percentages and ratios used herein are by weight of the total composition and all measurements made are at about 25° C. and about atmospheric pressure, unless otherwise designated.

All temperatures are in degrees Celsius unless specified otherwise. As used herein, “comprising” means the elements recited, or their equivalents in structure or function, plus any other element or elements which are not recited. The terms “having” and “including” are also to be construed as open ended. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Certain specific aspects and embodiments of the present application will be explained in greater detail with reference to the following examples, which are provided only for purposes of illustration and should not be construed as limiting the scope of the application in any manner.

Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Preparation of Amorphous Form of Daclatasvir Dihydrochloride

### Example 1

600 mg of daclatasvir dihydrochloride was dissolved in 10 ml of methanol. The solution was filtered to remove the undissolved particles and the filtrate was evaporated under 4 torr vacuum pressure at 55° C. After distillation the solid was dried at 55° C. for 2 hours and 15 minutes. Yield: 412 mg

### Example 2

600 mg of daclatasvir dihydrochloride was dissolved in a mixture of methanol (14.25 ml) and acetone (0.75 ml). The solution was filtered to remove the undissolved particles and the filtrate was evaporated under 4 torr vacuum pressure at 55° C. After evaporation the solid was dried at 55° C. for 2 hours and 15 minutes.

Yield: 460 mg

### Example 3

600 mg of daclatasvir dihydrochloride was dissolved in a mixture of methanol (14.25 ml) and acetic acid (0.75 ml). The solution was filtered to remove the undissolved particles and the filtrate was evaporated under 4 torr vacuum pressure at 55° C. After evaporation the solid was dried at 55° C. for 1 hours and 20 minutes.

Yield: 460 mg

### Example 4

2.5 gms of daclatasvir dihydrochloride was dissolved in 75 ml of methanol. The solution was filtered to remove undissolved particles. The solution was spray dried using following parameters.

Inlet temperature: 60° C.

Outlet Temperature: 40° C.

Aspirator: 70%

Feed pump speed: 30%

The obtained solid was dried at 55° C. for about 2 hours Yield: 1.0 g

### Example 5

200 mg of daclatasvir dihydrochloride was dissolved in 1.0 ml of methanol at 50° C. and added 2 ml of hexane to the solution. Then the mixture was poured to 15 ml of acetone. Obtained solid was dried at 55° C. for 1 hour in air tray dryer. Yield: 130 mg. The isolated amorphous form was found to be stable under ambient conditions for 24 hrs.

### Example 6

### Preparation of a Solid Dispersion Comprising an Amorphous Form of Daclatasvir Dihydrochloride and Copovidone

a) 500 mg of daclatasvir dihydrochloride and 500 mg of copovidone was dissolved in 15 ml of methanol. The solution was filtered to remove undissolved particles and the filtrate was evaporated under 4 torr vacuum pressure at 55° C. After evaporation the solid was dried at 55° C. for 1 hour and 30 minutes under vacuum.
b) Added 500 mg syloid to the above solid and mixed homogeneously.

### Example 7

### Preparation of a Solid Dispersion Comprising an Amorphous Form of Daclatasvir Dihydrochloride and PVP K-30

a) 500 mg of daclatasvir dihydrochloride and 500 mg of PVP k-30 was dissolved in 15 ml of methanol. The solution was filtered to remove undissolved particles and the filtrate was evaporated under 4 torr vacuum pressure at 55° C. After evaporation the solid was dried at 55° C. for 1 hour and 30 minutes under vacuum.
b) Added 500 mg syloid to the above solid and mixed homogeneously.

### Example 8

### Preparation of a Solid Dispersion Comprising an Amorphous Form of Daclatasvir Dihydrochloride and HPC

a) 500 mg of daclatasvir dihydrochloride and 500 mg of HPC was dissolved in 30 ml of methanol. The solution was filtered to remove undissolved particles and the filtrate was evaporated under 4 torr vacuum pressure at 55° C. After evaporation the solid was dried under vacuum at 55° C. for about 1 hour 45 minutes.
b) Added 500 mg syloid to the above solid and mixed homogeneously.

1. Daclatasvir dihydrochloride of Formula I in solid amorphous form.

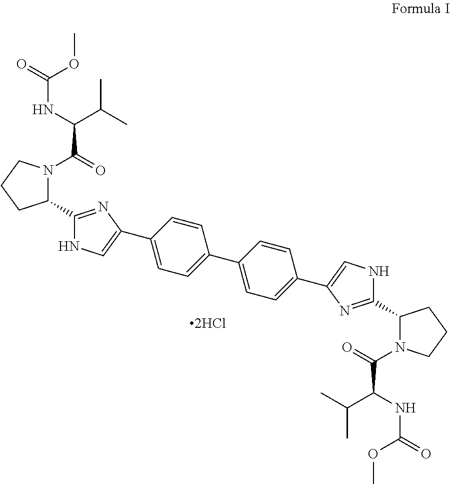 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)cc3)cc2)c[nH]1)C(C)C]

2. A process for preparing amorphous form of Daclatasvir dihydrochloride comprising:
a) providing a solution of Daclatasvir dihydrochloride in a solvent;
b) removing the solvent from the solution obtained in step a), and
c) recovering amorphous form of Daclatasvir dihydrochloride.

3. The process of claim 2 wherein suitable solvent in step a) is selected from alcohols, esters, ketones, hydrocarbons, water, acetic acid or mixtures thereof.

4. The process of claim 2 wherein suitable solvent in step a) is methanol.

5. The process of claim 2 wherein removal of solvent in step b) is affected by evaporation, freeze drying, spray drying, lyophilization, or any combination thereof.

6. A process for preparing amorphous form of daclatasvir dihydrochloride, comprising:
a) providing a solution of daclatasvir dihydrochloride in one or more solvents comprising methanol;
b) treating a solution obtained in step (a) with an anti-solvent and
c) recovering amorphous form of daclatasvir dihydrochloride.

7. The process of claim 6, wherein anti-solvent in step b) is selected from the group consisting of ketones, esters, hydrocarbons, ethers, water or mixtures thereof.

8. A solid dispersion comprising amorphous Daclatasvir dihydrochloride in a dispersing agent.

9. The solid dispersion of claim 8, wherein the dispersing agent comprises hydroxypropyl methyl cellulose (HPMC), Polyvinyl pyrrolidone (PVP), Co-povidone, Colloidal silicon dioxide and the like.

10. A pharmaceutical composition comprising solid dispersion of claim 8.

11. A process for preparing a solid dispersion comprising an amorphous form of daclatasvir dihydrochloride and one or more pharmaceutically acceptable carriers, comprising;
a) providing a solution comprising daclatasvir dihydrochloride and one or more pharmaceutically acceptable carriers,
b) removing solvent from a solution obtained in step (a) and
c) recovering a solid dispersion comprising an amorphous form of daclatasvir dihydrochloride and one or more pharmaceutically acceptable carriers.
